(19) United States
(12) Patent Application Publication
(10) Pub. No.: US 2015/0212087 A1
(43) Pub. Date: Jul. 30, 2015

(54) Detection of the ERalpha/Src/PI3K Complex as Predictive Marker in Breast Cancer

(71) Applicants: Centre Leon Berard, Lyon (FR); Centre National de la Recherche Scientifique (CNRS), Paris (FR); Institut National de la Sante et de la Recherche Medicale (INSERM), Paris (FR); Universite Claude Bernard Lyon 1, Villeurbanne (FR)

(72) Inventors: Muriel Le Romancer-Cherifi, Sainte Foy Les Lyon (FR); Olivier Tredan, Ecully (FR); Isabelle Treilleux, Lyon (FR)

(21) Appl. No.: 14/417,269

(22) PCT Filed: Jul. 26, 2013
(86) PCT No.: PCT/EP2013/065782

§ 371 Date: Jan. 26, 2015

Related U.S. Application Data
(60) Provisional application No. 61/676,418, filed on Jul. 27, 2012.

(30) Foreign Application Priority Data
Jul. 27, 2012 (EP) 12305918.0

(51) Int. Cl. G01N 33/574

(52) U.S. Cl. CPC G01N 33/57415; G01N 2800/52; G01N 2333/91215

(57) ABSTRACT

Detection of the ERα/Src/PI3K complex by a Proximity ligation assay as prognostic and theranostic marker in breast cancer.

The present invention relates to methods for determining the prognosis of a breast cancer, to methods for identifying a breast cancer likely to respond to treatment with anti-estrogens and to treatment with Src/PI3K inhibitors and to methods of treatment of a breast cancer patient.

Due to the major role that ERα plays in the development and progression of breast cancer, the estrogen signaling pathway has been studied in depth. Current endocrine therapies for breast cancer are mainly based on targeting the ERα signaling pathway: reducing estrogen abundance with aromatase inhibitor, antagonizing ERα function with tamoxifen and raloxifene or down-regulating ERα expression with fulvestrant. However, resistance to endocrine therapies is one of the major barriers to the successful treatment of breast cancer (Musgrove and Sutherland, 2009). There is a real need to find markers predicting resistance to treatment. So far, ERα expression in the nucleus is currently the only known biomarker of response to endocrine therapy. As a consequence, non-genomic ERα signaling has never been assessed in clinical practice. We previously reported that methylation of ERα on arginine 260, via the arginine methyltransferase PRMT1, is a prerequisite for its association with Src, PI3K and the Focal Adhesion Kinase (FAK) as well as activation of its downstream effector Akt (Le Romancer M. et al., 2010; Le Romancer M. et al., 2008). Moreover, we have shown that this modification occurs in the cytoplasm of normal breast epithelial cells and is highly expressed in a subset of breast tumors. However, we did not find any correlation between ERα methylation and patient's survival in a serie of 164 breast cancers (Le Romancer M. et al., 2008).

The tyrosine kinase Src has also been considered as a potential target and Src inhibitors like dasatinib or bosutinib have been tested in phase II clinical trials (Araujo and Logothetis, 2010). However, so far the effects have been quite disappointing. In fact, dasatinib used as a single agent has limited activity in patients with TNBC, or patients with heavily treated metastatic breast cancer and it advances ERα-positive tumors. However, in vitro studies show that combining anti-estrogen and Src inhibitor enhances growth inhibition. Moreover, clinical trials are ongoing to combine dasatinib with other therapies.

However, even if some clinical studies give satisfactory results, there remains a real need to identify biomarkers that will predict which patients could benefit from these inhibitors either alone or in combination.

### SUMMARY

A first object of the present invention is a method for determining the prognosis of a breast cancer in a patient comprising the following steps:
a) Measuring the level of expression of ERα/Src/PI3K protein complexes in the cytoplasm of cancer cells from a breast tumour sample previously taken from said patient,
b) Classifying the breast cancer as having a poor prognosis if ERα/Src/PI3K protein complexes are overexpressed in the cytoplasm of said cancer cells.

In preferred embodiments, the breast cancer has been classified as ER+.

Preferably, the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

In one embodiment, in step b) the level of expression of ERα/Src/PI3K protein complexes is compared to the median level of expression of ERα/Src/PI3K protein complexes in healthy breast tissue.

In another embodiment, in step b) the level of expression of ERα/Src/PI3K protein complexes is compared to the median level of expression of ERα/Src/PI3K protein complexes in breast tumour samples.

Another object of the present invention is a method for identifying a breast cancer likely to respond to treatment with anti-estrogens and to treatment with Src inhibitors and/or PI3K inhibitors comprising the following steps:
a) Measuring the level of expression of ERα/Src/PI3K protein complexes in the cytoplasm of cancer cells from a breast tumour sample previously taken from said patient,
b) Classifying the breast cancer as likely to respond to treatment with anti-estrogens and to treatment with Src inhibitors and/or PI3K inhibitors if the ERα/Src/PI3K protein complex is overexpressed in the cytoplasm of said cancer cells.

In one embodiment, the breast cancer has been classified as ER+.

In another embodiment, the breast cancer has been classified as ER−.

Preferably, the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

The present invention further relates to a composition comprising a Src inhibitor and/or a PI3K inhibitor for use in methods of treatment of a breast cancer patient, wherein said use comprises the following steps:
a) Measuring the level of expression of ERα/Src/PI3K protein complexes in the cytoplasm of cancer cells from a breast tumour sample previously taken from said patient,
b) Selecting a breast cancer patient having a breast cancer tumour in which the ERα/Src/PI3K protein complex is overexpressed in the cytoplasm of said cancer cells,
c) Administering to said patient a therapeutically effective amount of a Src inhibitor and/or a PI3K inhibitor.

Preferably, in step c) the Src inhibitor and/or the PI3K inhibitor are administered in combination with an anti-estrogen.

Preferably, in step a) the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

In one embodiment, the breast cancer has been classified as ER+.

In another embodiment, the breast cancer has been classified as ER−.

### DETAILED DESCRIPTION OF THE INVENTION

The methylated form of ERα is detected in the cytoplasm of breast cells and is highly expressed in a subset of breast tumours. However, this level of methylation is not a prognostic marker of breast cancer. The present invention remarkably shows that overexpression of the ERα/Src/PI3K complex in the cytoplasm of breast tumour cells is associated with a poor prognosis in breast cancer patients.

The present invention is related to methods for determining the prognosis of a breast cancer in a patient.

The present invention is also directed to methods for identifying a breast cancer likely to respond to treatment with anti-estrogens and/or to treatment with Src inhibitors and/or PI3K inhibitors.

Preferably, the methods of the present invention are in vitro methods.

The term “ERα/Src/PI3K complex” refers to the association of three different proteins in a protein complex. This protein complex has been described in the cytoplasm of breast cells and in the cytoplasm of breast tumour cells.

The term “ERα” refers to human estrogen receptor protein encoded by the ESR1 gene (HGNC ID=HGNC:3467).

The term “Src” refers to human tyrosine kinase encoded by the SRC gene (HGNC:11283).

The term “PI3K” refers to Phosphatidylinositol 3-kinase regulatory subunit encoded by the PIK3R1 gene (NM_{—}181523).

The present invention is based on the detection of the ERα/Src/PI3K protein complex in the cytoplasm of breast tumour cells. More preferably, the present invention is based on the detection of the interaction between the proteins ERα and SRC and/or on the detection of the interaction between the proteins ERα and PI3K in the cytoplasm of breast tumour cells.

The interaction or the formation of a complex between these proteins may be detected and measured by any appropriate method. Various methods are known to the skilled person to detect and measure interactions between proteins leading to the formation of protein complexes. In the present invention, the level of expression of ERα/Src/PI3K protein complexes is preferably carried out by detecting ERα/Src protein complexes by a Proximity Ligation Assay (PLA) using anti-ERα and anti-Src antibodies and/or the level of expression of ERα/Src/PI3K protein complexes is measured by detecting ERα/PI3K protein complexes by a Proximity Ligation Assay using anti-ERα and anti-PI3K antibodies.

Any appropriate antibodies may be used in the Proximity Ligation Assays according to methods known to the skilled person. Antibodies against ERα, Src and PI3K are available from various commercial sources. For PI3K, antibodies commercialized by Abcam® under the reference Ab22653 may be used. For Src (B12), antibodies commercialized by Santa Cruz® under the reference Sc-8056 may be used. For ERα, antibodies commercialized by Santa Cruz® under the reference Sc-542 may be used.

The term “sample” refers to any biological sample obtained/taken from a patient including a tissue sample, a cell sample or a tumour sample. In the present invention, the sample is a breast tumour sample containing cancer or tumour cells. In preferred embodiments, the breast tumour sample is a primary tumour sample.

The term “cancer” refers to any disease in which a group of cells displays uncontrolled growth/proliferation, invasion and sometimes metastasis.

In the methods of the present invention, the level of expression of the ERα/Src/PI3K complex is preferably compared to a control sample. In some embodiments, the control sample is the median level of expression of the ERα/Src/PI3K complex observed in a healthy breast tissue. More advantageously, the control sample is the median level of expression of ERα/Src/PI3K complex in tumour samples taken from patients having breast cancers and more preferably in primary breast tumor samples.

In another embodiment, the control sample is the median level of expression of the ERα/Src/PI3K complex in ER+(ER-positive) breast tumours and more preferably in ER+ primary breast tumours.

In another embodiment, the control sample is the median level of expression of the ERα/Src/PI3K complex in ER− (ER-negative) breast tumours and more preferably in ER-primary breast tumours.

ER status is a conventional marker of breast cancer based on the presence or absence of the estrogen receptor (ERα). Generally, ER+ breast cancers are classified as having a “better” prognosis. Receptor status may be determined by classical methods well known to the skilled person. Breast cancers of the ER+ subclass are considered as having a “favorable” prognosis although a number of these patients will experience a recurrence of their breast cancer. Breast tumours or breast cancer patients belonging to the ER+ subclass are usually selected or are considered suitable for anti-estrogen/hormone therapy.

The present invention is directed to methods for identifying ER+ breast cancer which have a poor prognosis and which are more likely to respond to treatment with both anti-estrogens and Src inhibitors and/or PI3K inhibitors.

The present invention is also directed to methods for identifying ER− breast cancer which have a poor prognosis and which are more likely to respond to treatment with both anti-estrogens and Src inhibitors and/or PI3K inhibitors. ER− breast cancers are usually not considered likely to respond to anti-estrogen/hormone therapy.

In the present invention the breast cancer patient may previously have undergone surgery for breast cancer to remove breast tumour.

In breast cancer, PI3K inhibitors are typically used in combination with hormone/endocrine therapy.

In hormone-receptor positive cancers, the hormone estrogen promotes the growth of breast cancer cells. Hormone therapy aims to block the effect of estrogen/progesterone or lower its levels in order to treat breast cancer. Some hormonal treatments are for example targeted at the estrogen receptor (ER). Hormone therapeutic drugs include compounds which block the estrogen receptor such as tamoxifen, raloxifen and fulvestrant. Hormone therapeutic drugs also include aromatase inhibitors blocking the synthesis of estrogen such as exemestane, anastrozole and letrozole.

Hormone therapy may be used to help reduce the risk of the cancer coming back after surgery, but it may also be used for breast cancer that has spread or come back after treatment. This therapy may therefore be used in an adjuvant setting after breast surgery or in a metastatic setting.

The terms “administered in combination” refers to administration to a same patient of different therapies over a period of time. Administration of different therapies in combination may occur simultaneously or separately. In the present invention, Src inhibitors, PI3K inhibitors and hormone therapy may be administered simultaneously or separately. Preferably, Src inhibitors, PI3K inhibitors and hormone therapy are administered separately.

In the present invention, Src inhibitors and/or PI3K inhibitors may be administered in combination with therapeutic drugs blocking the estrogen receptor or in combination with aromatase inhibitors. In preferred embodiments, Src inhibitors and/or PI3K inhibitors are administered in combination with tamoxifen, raloxifen, fulvestrant, exemestane, anastrozole and letrozole.

The term “anti-estrogens” refers to hormone therapy and to therapeutic drugs blocking the estrogen receptor or to aromatase inhibitors. Preferably, “anti-estrogens” refers to tamoxifen, raloxifen, fulvestrant, exemestane, anastrozole and letrozole.

The terms “Src inhibitors” refers to compounds inhibiting Src family tyrosine kinase. Src inhibitors include dasatinib, bosutinib and saracatinib.

The terms “PI3K inhibitor” refers to compounds inhibiting Phosphoinositide 3-kinase enzyme. “PI3K inhibitors” include Perifosine, CAL101, PX-866, BEZ235, SF1126, INK1117, IPI-145, GDC-0941, BKM120, XL147, XL765, Palomid 529, GSK1059615, ZSTK474 and PWT33597.

Src inhibitors and/or PI3K inhibitors may be administered in a metastatic setting or in an adjuvant setting. Adjuvant therapy is defined as a treatment given after the primary therapy to prevent that the cancer will come back or spread. Adjuvant therapy is typically applied after breast cancer surgery.

In the present invention, breast cancer patients are selected which are more likely to respond to treatment with Src inhibitors and/or PI3K inhibitors. The identification of breast tumours/breast cancer patients which are more likely to benefit from treatment with Src inhibitors and/or PI3K inhibitors promotes a broader use of Src inhibitors and/or PI3K inhibitors in an adjuvant setting.

A first object of the present invention is a method for determining the prognosis of a breast cancer in a patient comprising the following steps:
a) Measuring the level of expression of ERα/Src/PI3K protein complexes in the cytoplasm of cancer cells from a breast tumour sample previously taken from said patient,
b) Classifying the breast cancer as having a poor prognosis if ERα/Src/PI3K protein complexes are overexpressed in the cytoplasm of said cancer cells.

The present invention also relates to methods for determining the prognosis of a breast cancer in a patient comprising the following steps:
a) Obtaining a breast tumour sample from said patient,
b) Measuring the level of expression of ERα/Src/PI3K protein complexes in the cytoplasm of cancer cells from said breast tumour sample,
c) Classifying the breast cancer as having a poor prognosis if ERα/Src/PI3K protein complexes are overexpressed in the cytoplasm of said cancer cells.

The present invention also relates to methods of determining the prognosis of a patient diagnosed with breast cancer.

A “prognosis” is the likely course and outcome of a disease. The prognosis may include the likelihood of complications of the cancer, of metastasis, of spread, probable outcome of the cancer, likelihood of recovery, disease free survival, overall survival rate and/or overall death rate. Preferably, it is the probability that a patient will recover or have a recurrence/relapse of the cancer. This information is useful to the patient but also to the physician in determining the most effective course of treatment. A determination of the likelihood for a cancer relapse or of the likelihood of metastasis can assist the physician in determining whether a more conservative or a more radical approach to therapy should be taken. Prognosis provides for selection and classification of patients who are predicted to benefit from a given therapeutic regimen.

The methods of the present invention provide prognosis for breast cancer after it has been diagnosed and/or during therapeutic treatment.

Over-expression or high expression levels of ERα/Src/PI3K protein complexes in the cytoplasm of cancer cells are characteristic of breast tumors having a poor prognosis for disease-free survival (DFS). Over-expression of this protein complex in the cytoplasm breast cancer cells is statistically significantly correlated with increased disease recurrence and worse prognosis.

The present invention relates to a method for identifying a breast cancer and/or a breast tumor prone to recur and/or a breast cancer and/or a breast tumor having or prone to develop an invasive or metastatic phenotype. More specifically, the present invention relates to a method for identifying a breast cancer and/or a breast tumor prone to recur and/or a breast cancer and/or a breast tumor having or prone to develop an invasive or metastatic phenotype.

The term “DFS” refers to Disease Free Survival and is defined as the percentage of patients staying free of disease progression during a period of time. In this case, the Kaplan-Meier curve represents the x % of patients staying free of disease progression after y amount of time.

The DFS in a patient diagnosed with a breast cancer exhibiting over-expression or high expression levels of ERα/Src/PI3K protein complexes in the cytoplasm of cancer cells, is reduced compared to a patient who has not an overexpression of the ERα/Src/PI3K protein complexes in the cytoplasm of cancer cells.

In preferred embodiments, the breast cancer has been classified as ER+. ER status may have been determined previously or may be determined at the same time or after measurement of the level of expression of ERα/Src/PI3K protein complexes in the cytoplasm of cancer cells from the breast tumour sample. Preferably, the breast cancer has been previously classified as ER+ and the measurement of the level of expression of ERα/Src/PI3K protein complexes is intended to determine whether the breast cancer patient has a decreased DFS in spite of a favourable ER status.

Preferably, the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

In a first embodiment, the level of expression of ERα/Src/PI3K protein complexes in step b) is compared to the median level of expression of ERα/Src/PI3K protein complexes in healthy breast tissue.

In a second embodiment, the level of expression of ERα/Src/PI3K protein complexes in step b) is compared to the median level of expression of ERα/Src/PI3K protein complexes in breast tumour samples.

A second object of the present invention is a method for identifying a breast cancer likely to respond to treatment with anti-estrogens and to treatment with Src inhibitors and/or PI3K inhibitors comprising the following steps:
a) Measuring the level of expression of ERα/Src/PI3K protein complexes in the cytoplasm of cancer cells from a breast tumour sample previously taken from said patient,
b) Classifying the breast cancer as likely to respond to treatment with anti-estrogens and to treatment with Src inhibitors and/or PI3K inhibitors if the ERα/Src/PI3K protein complex is overexpressed in the cytoplasm of said cancer cells.

The present invention also relates to a method for identifying a breast cancer likely to respond to treatment with anti-estrogens and to treatment with Src inhibitors and/or PI3K inhibitors comprising the following steps:
a) Obtaining a breast tumour sample from said patient,
b) Measuring the level of expression of ERα/Src/PI3K protein complexes in the cytoplasm of cancer cells from said breast tumour sample,
c) Classifying the breast cancer as likely to respond to treatment with anti-estrogens and to treatment with Src inhibitors and/or PI3K inhibitors if the ERα/Src/PI3K protein complex is overexpressed in the cytoplasm of said cancer cells.

In another embodiment, the present invention is also directed to a method for identifying a breast cancer patient suffering from a tumour likely to respond to treatment with anti-estrogens and to treatment with Src inhibitors and/or PI3K inhibitors.

Anti-estrogens, Src inhibitors and/or PI3K inhibitors may be administered simultaneously or separately over a period of time.

In a first embodiment, the breast cancer has been classified as ER+. In a second embodiment, the breast cancer has been classified as ER−. The receptor status of the breast cancer patient may have been determined before, during or after measurement of the level of expression of ERα/Src/PI3K protein complexes in the cytoplasm of cancer cells from said breast tumour sample. In a preferred embodiment, the ER receptor status has been determined before measurement of the level of expression of ERα/Src/PI3K protein complexes in the cytoplasm of cancer cells.

Preferably, the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

In a first embodiment, the level of expression of ERα/Src/PI3K protein complexes in step b) is compared to the median level of expression of ERα/Src/PI3K protein complexes in healthy breast tissue.

In a second embodiment, the level of expression of ERα/Src/PI3K protein complexes in step b) is compared to the median level of expression of ERα/Src/PI3K protein complexes in breast tumour samples.

Another object of the present invention is a composition comprising an anti-estrogen for use in methods of treatment of a breast cancer patient wherein said use comprises the following steps:
a) Measuring the level of expression of ERα/Src/PI3K protein complexes in the cytoplasm of cancer cells from a breast tumour sample previously taken from said patient,
b) Selecting a breast cancer patient having a breast cancer tumour in which the ERα/Src/PI3K protein complex is overexpressed in the cytoplasm of said cancer cells,
c) Administering to said patient a therapeutically effective amount of anti-estrogen.

In preferred embodiments, anti-estrogens are administered in step c) in combination with a Src inhibitor and/or a PI3K inhibitor.

The breast cancer patient may previously have been classified as ER+ or ER−.

In preferred embodiments, the breast cancer patient has previously been classified as ER−.

The present invention is also related to a method of treatment of a breast cancer patient wherein said use comprises the following steps:
a) Obtaining a breast tumour sample from said patient,
b) Measuring the level of expression of ERα/Src/PI3K protein complexes in the cytoplasm of cancer cells from a breast tumour sample from said patient,
c) Selecting a breast cancer patient having a breast cancer tumour in which the ERα/Src/PI3K protein complex is overexpressed in the cytoplasm of said cancer cells,
d) Administering to said patient a therapeutically effective amount of anti-estrogen.

In preferred embodiments, anti-estrogen is administered in step d) in combination with a Src inhibitor and/or a PI3K inhibitor.

The breast cancer patient may previously have been classified as ER+ or ER−.

In preferred embodiments, the breast cancer patient has previously been classified as ER−.

Another object of the present invention is a composition comprising a Src inhibitor and/or a PI3K inhibitor for use in methods of treatment of a breast cancer patient wherein said use comprises the following steps:
a) Measuring the level of expression of ERα/Src/PI3K protein complexes in the cytoplasm of cancer cells from a breast tumour sample previously taken from said patient,
b) Selecting a breast cancer patient having a breast cancer tumour in which the ERα/Src/PI3K protein complex is overexpressed in the cytoplasm of said cancer cells,
c) Administering to said patient a therapeutically effective amount of Src inhibitor and/or PI3K inhibitor.

In preferred embodiments, Src inhibitor and/or PI3K inhibitor is administered in step c) in combination with anti-estrogen.

The breast cancer patient may previously have been classified as ER+ or ER−. In preferred embodiments, the breast cancer patient has previously been classified as ER+.

The present invention is also related to a method of treatment of a breast cancer patient wherein said method comprises the following steps:
a) Obtaining a breast tumour sample from said patient,
b) Measuring the level of expression of ERα/Src/PI3K protein complexes in the cytoplasm of cancer cells from a breast tumour sample from said patient,
c) Selecting a breast cancer patient having a breast cancer tumour in which the ERα/Src/PI3K protein complex is overexpressed in the cytoplasm of said cancer cells,
d) Administering to said patient a therapeutically effective amount of Src inhibitor and/or PI3K inhibitor.

In preferred embodiments, Src inhibitor and/or PI3K inhibitor is administered in step d) in combination with anti-estrogens.

The breast cancer patient may previously have been classified as ER+ or ER−. In preferred embodiments, the breast cancer patient has previously been classified as ER+.

Preferably, the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

In a first embodiment, the level of expression of ERα/Src/PI3K protein complexes in step b) is compared to the median level of expression of ERα/Src/PI3K protein complexes in healthy breast tissue.

In a second embodiment, the level of expression of ERα/Src/PI3K protein complexes in step b) is compared to the median level of expression of ERα/Src/PI3K protein complexes in breast tumour samples.

### FIGURES

FIG. 1: In situ PLA detection of endogenous ERα/PI3K and ERα/Src interactions in MCF-7 cells.

FIG. 2: Time course of ERα/PI3K and ERα/Src interactions in MCF-7 cells.

FIG. 3: In Situ PLA detection of ERα/PI3K and ERα/Src interactions upon tamoxifen treatment.

FIG. 4: Control of ERα/PI3K and ERα/Src interactions using siERα.

FIG. 5: In situ PLA detection of endogenous FAK/Src and FAK/ERα interactions in MCF-7 cells.

FIG. 6: Control of ERα/PI3K and ERα/Src interactions using siPRMT1.

FIG. 7: In Situ PLA detection of ERα/PI3K and ERα/Src interactions upon PP1 and LY294002 treatment.

FIG. 8: In Situ PLA detection of ERα/PI3K and ERα/Src interactions upon a peptide treatment.

FIG. 9: In situ PLA detection of endogenous ERα/PI3K and ERα/Src interactions In human breast cancer cell lines.

FIG. 10: ERα/Src/PI3K complex expression in human normal breast tissue (n=3).

FIG. 11: Distribution of ERα/Src, ERα/PI3K and ERα/mERα data.

FIG. 12: Correlation analysis between the different markers and p-Akt.

FIG. 13: Distribution of clinical parameters according to groups of ERα/Src expression.

FIG. 14: Distribution of clinical parameters according to groups of ERα/PI3K expression.

FIG. 15: Distribution of clinical parameters according to groups of ERα/mERα expression.

FIG. 16: Kaplan Meier estimates of DFS by ERα/Src expression groups.

FIG. 17: Multivariate Cox model integrating ERα/Src.

FIG. 18: Kaplan-Meier estimates of OS by ERα/Src expression groups.

FIG. 19: Kaplan Meier estimates of DFS by ERα/PI3K expression groups.

FIG. 20: Kaplan-Meier estimates of OS by ERα/PI3K expression groups.

FIG. 21: Multivariate Cox model integrating ERα/PI3K.

FIG. 22: Kaplan-Meier estimates of patient's outcome for mERα/ERα expression groups Global population (with a cut off at 3 spots per cell) for DFS (A) and for OS (B).

### EXAMPLES

### Example 1

### Detection of Endogenous ERα/PI3K and ERα/Src Interactions in Human Breast Tumor Cells

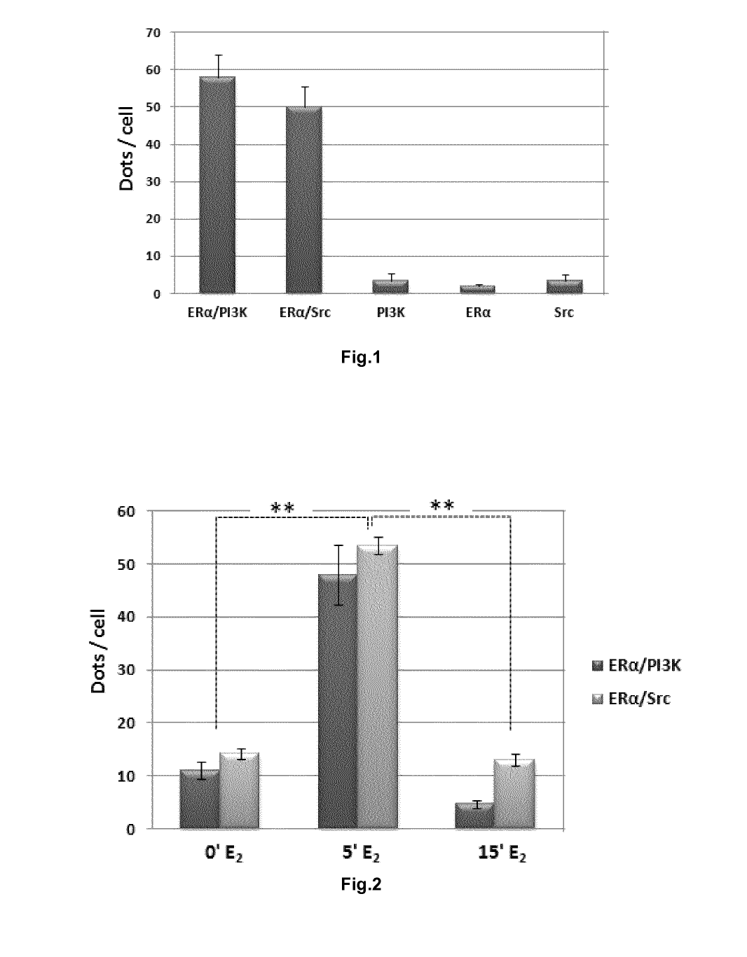

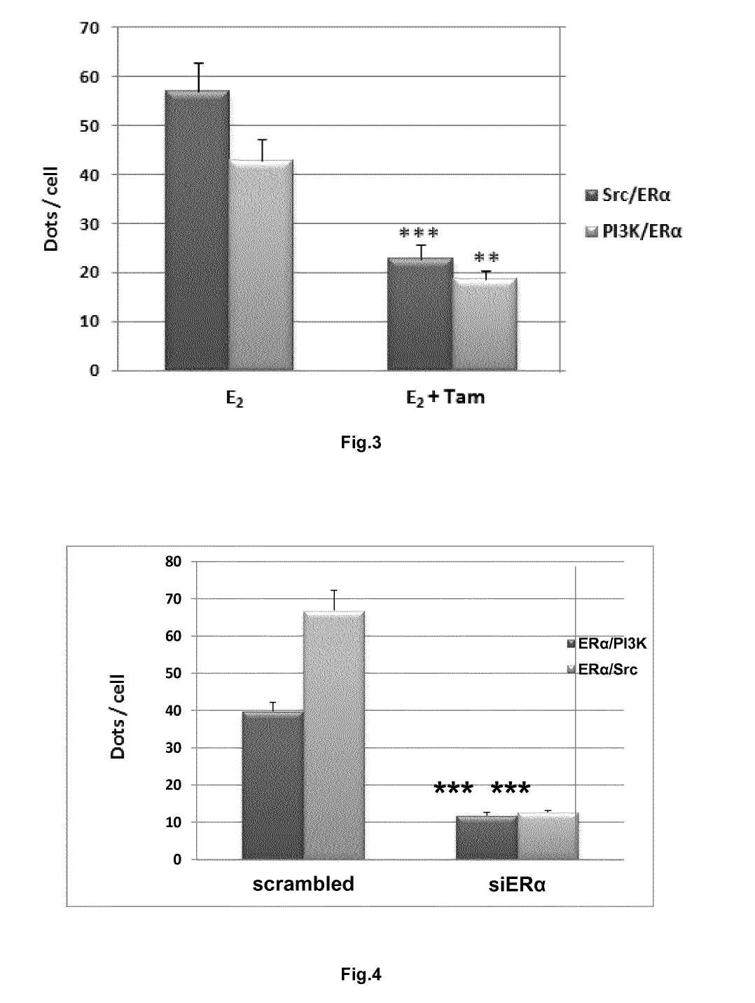

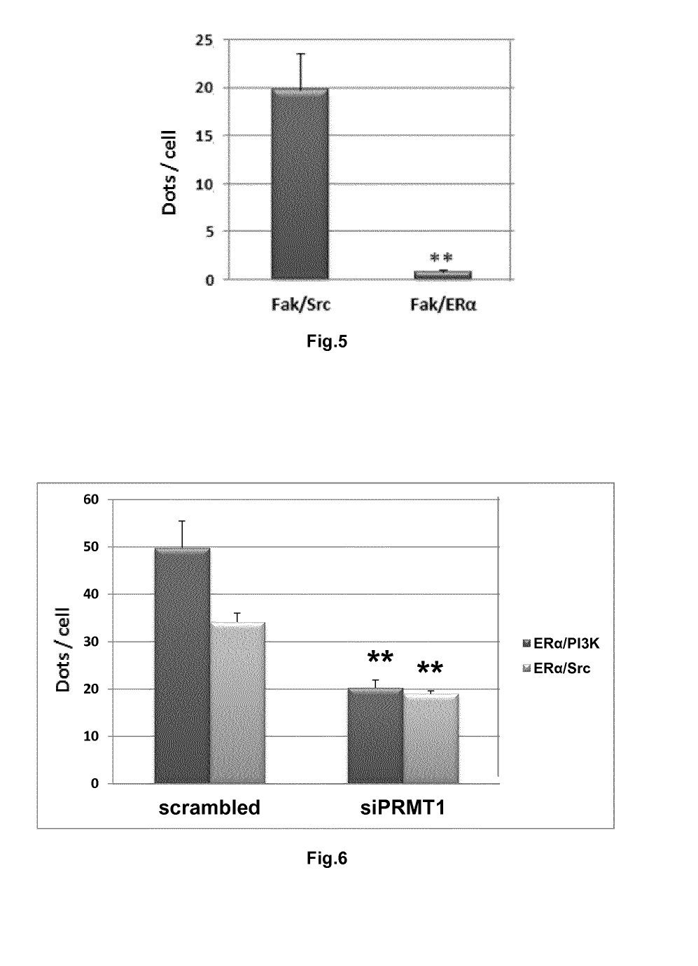

Castoria et al. reported that estrogen rapidly triggers the interaction of ERα with Src and PI3K in MCF-7 cells, forming a complex involved in estrogen nongenomic-induced cell proliferation. This result has largely been confirmed by others in several breast cell lines as well as in other tissues. However, all of these results were obtained by immunoprecipitation in cell lines that did not allow the visualization of interactions between proteins. Therefore the physiological relevance of this signaling pathway remains questionable. To date, immuno fluorescence analysis of the complex has been impeded by the fact that only a small population of ERα interacts with Src and PI3K. To circumvent this problem, we used a newly developed technique, the Proximity Ligation Assay. Protein-protein interactions were sensitively and specifically demonstrated using pairs of proximity probes and detected by in situ circular amplification, with each red dot representing an interaction (Soderberg et al., 2006). We investigated the ERα/PI3K interaction in the human breast tumor cell line MCF-7, using a rabbit anti-ERα together with a mouse anti-p85 antibody. The ERα/Src interaction was detected using the same anti-ERα together with a mouse anti-Src antibody. We found that ERα interacted with PI3K and Src in the cytoplasm of MCF-7 cells, as indicated by the presence of red dots for both antibody pairs. No dots were detected using only one antibody as confirmed by counting dots per 100 cells (FIG. 1, around 50 dots/cell versus less than 5). Importantly, the number of red dots increased after 5 min of estrogenic treatment then decreased after 15 min. This confirmed that upon estrogenic treatment, the formation of this complex is rapid and transitory (FIG. 2:)). As expected, we observed a decrease in the interaction between ERα/PI3K and ERα/Src in MCF-7 cells upon tamoxifen treatment (FIG. 3) and ERα knockdown (FIG. 4), validating the specificity of the above results. In addition, we performed a set of controls to further validate the specificity of PLA technology. We tested the interactions between ERα with two known ERα nuclear co-activators, SRC3 and p300. They were detected exclusively in the nucleus of MCF-7 cells, as expected. We previously identified that the Focal Adhesion kinase (FAK) is also recruited into the complex (Le Romancer M. et al., 2008) as confirmed by others (Sanchez et al., 2010). Therefore we studied the interaction of FAK with ERα by PLA. As seen in FIG. 5, although FAK interacts with Src, we did not detect any red dots for ERα/FAK interaction. This result is concordant with our previous data showing that the recruitment of FAK into the complex is mediated by its interaction with Src.

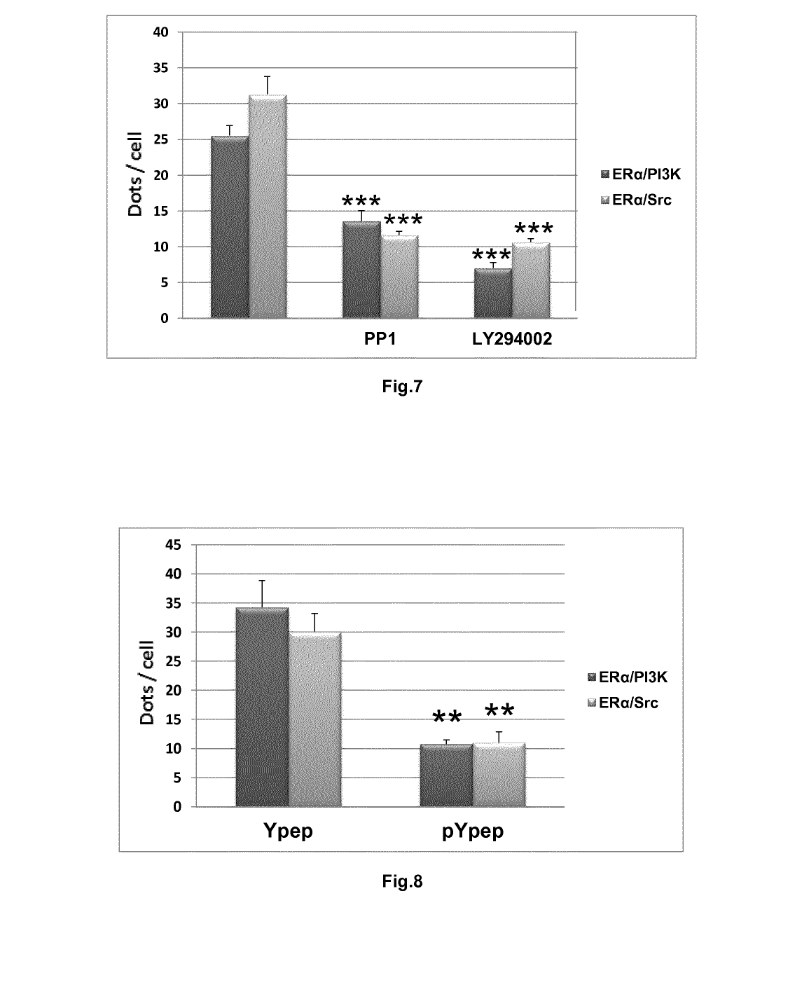

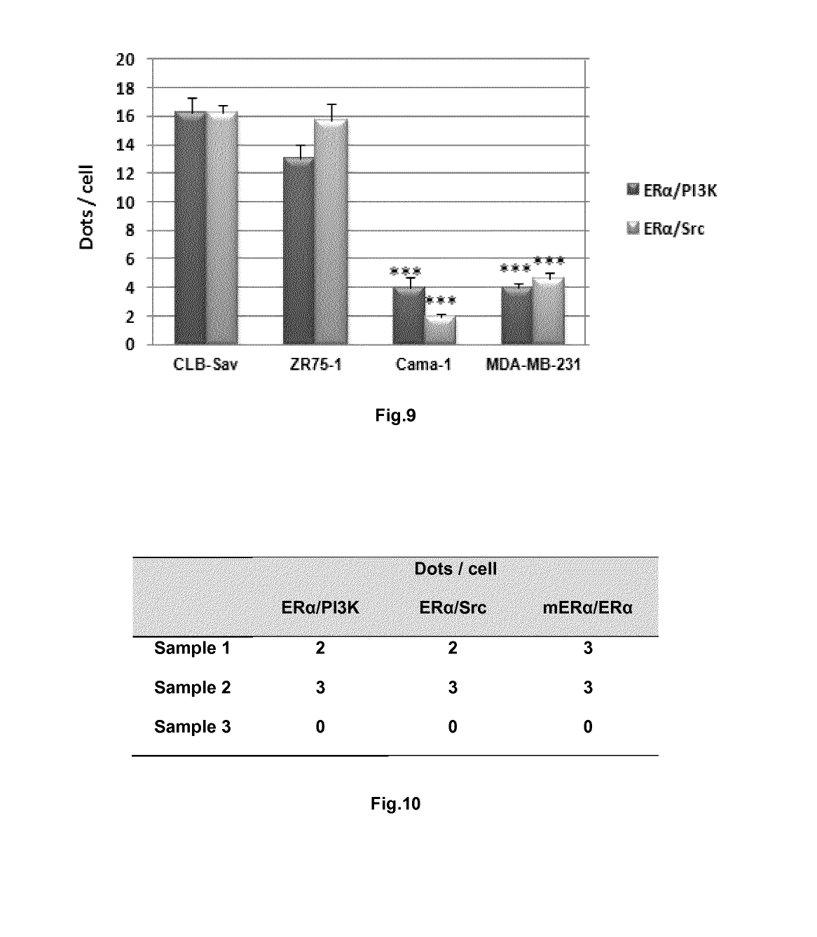

We previously showed that the formation of the ERα/PI3K/Src complex requires the methylation of ERα as well as the kinase activity of Src and PI3K (Le Romancer M. et al., 2008). Therefore we performed PLA analysis either using PRMT1 knockdown cells (FIG. 6) or after the addition of PP1 (Src inhibitor) or LY294002 (PI3K inhibitor) (FIG. 7). PLA analysis confirmed these results with a significant decrease of red dots. Furthermore, the group of Aurricchio found that a six-amino acid peptide (pYpep), that mimics the sequence around the phosphotyrosine residue in position 537 of the human ERα, disrupts ERα/Src interaction and estrogen-induced proliferation (Varricchio et al., 2007). Indeed, treatment with the phosphorylated peptide induced a notable disruption of the complex, visualized by both immunoprecipitation and PLA analysis (FIG. 8). Finally, we confirmed the interactions between ERα/PI3K and ERα/Src using the ERα positive cell lines CLB-SAV, ZR75.1 and Cama-1, and the ERα negative cell line MDA-MB-231. Both complexes were present in the cytoplasm of CLB-SAV and ZR75.1 cells but not in Cama-1 cells nor MDA-MB-231 cells. Formation of the complex was concordant with the methylation of ERα as we did not detect any estrogen-induced methylation in either MDA-MB-231 or in Cama-1 cells (FIG. 9).

All these in vitro data clearly validate PLA technology as a powerful tool to analyze ERα/PI3K and ERα/Src interactions.

### Example 2

### ERα Interacts with PI3K and Src in Normal Breast Samples

A crucial question about estrogen nongenomic signaling concerns its existence in physiology. To approach this issue, we firstly tested the presence of the ERα/Src/PI3K complex in 3 human normal breast samples from mammoplasty. Thus, we performed PLA experiments using the two previously described pairs of antibodies to study the ERα/Src and ERα/PI3K interactions. To correlate these interactions with the presence of methylated ERα we detected mERα by PLA using rabbit anti-ERα together with the mouse anti-mERα antibody (mERα/ERα). We detected ERα/PI3K ERα/Src and mERα/ERα expression in the cytoplasm of epithelial cells but not myoepithelial cells. The quantification of red dots revealed a low level expression of the complex. This was expected as ERα is faintly expressed in normal breast epithelial cells. We obtained similar results for all 3 mammary samples (FIG. 10).

### Example 3

### In Human Breast Cancers the Interaction of ERα with Both PI3K and Src, Correlates with ERα Methylation and Akt Activation

We next evaluated the presence of the ERα/PI3K and ERα/Src complexes as well as mERα/ERα expression in 175 invasive breast tumors. The signal for each protein couple varied in intensity from null to a very strong signal.

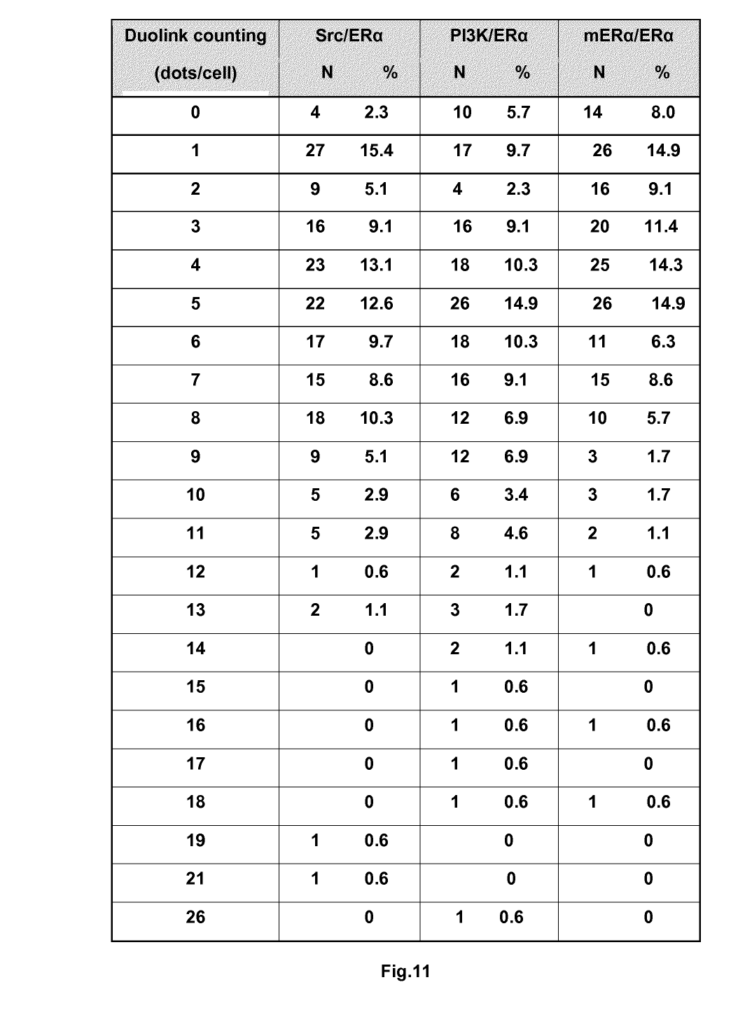

To perform these highly scaled experiments, we used a different PLA revelation kit which allowed the visualization of brown dots in bright field microscope. We also performed immunohistochemistry analysis using an anti-P-Akt antibody on the same tumor samples in order to confirm that ERα methylation triggers Akt activation. Results from these PLA experiments were quantified by counting at least 400 cells and expressed as the mean number of dots per cell as described in the material and methods section (See FIG. 11).

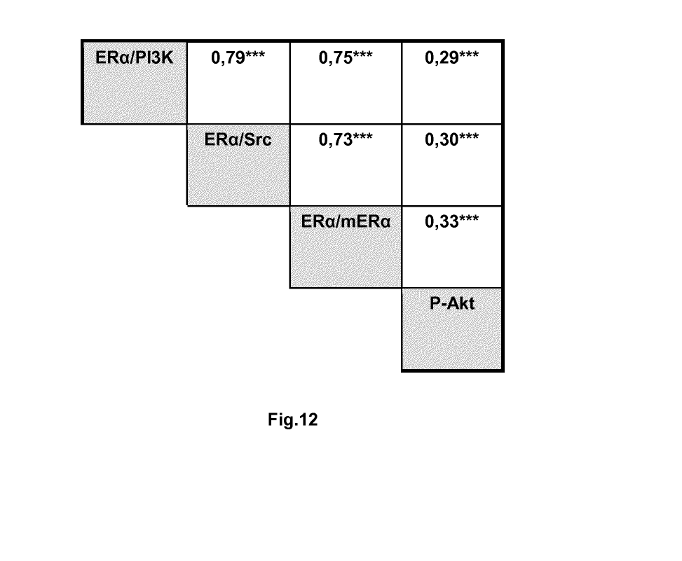

Interestingly, when we performed a correlation analysis between the different markers, we found significant correlations between ERα/PI3K, ERα/Src interactions and mERα expression (p<0.001) (FIG. 12). This confirms our hypothesis that mERα is responsible for forming the complex. We also discovered statistically significant correlations between each protein couple and P-Akt expression.

These data consistently demonstrate that ERα methylation is required for mediating the interaction of the estrogen receptor with Src and PI3K, which propagates the signal to downstream transduction cascades. Overexpression of mERα and the signaling complex can lead to the hyperactivation of Akt.

### Example 4

### Based on the Quantification of Dots Per Cell for Each Protein Couple, we

analyzed the association between their expression and clinical parameters for 175 breast tumors.

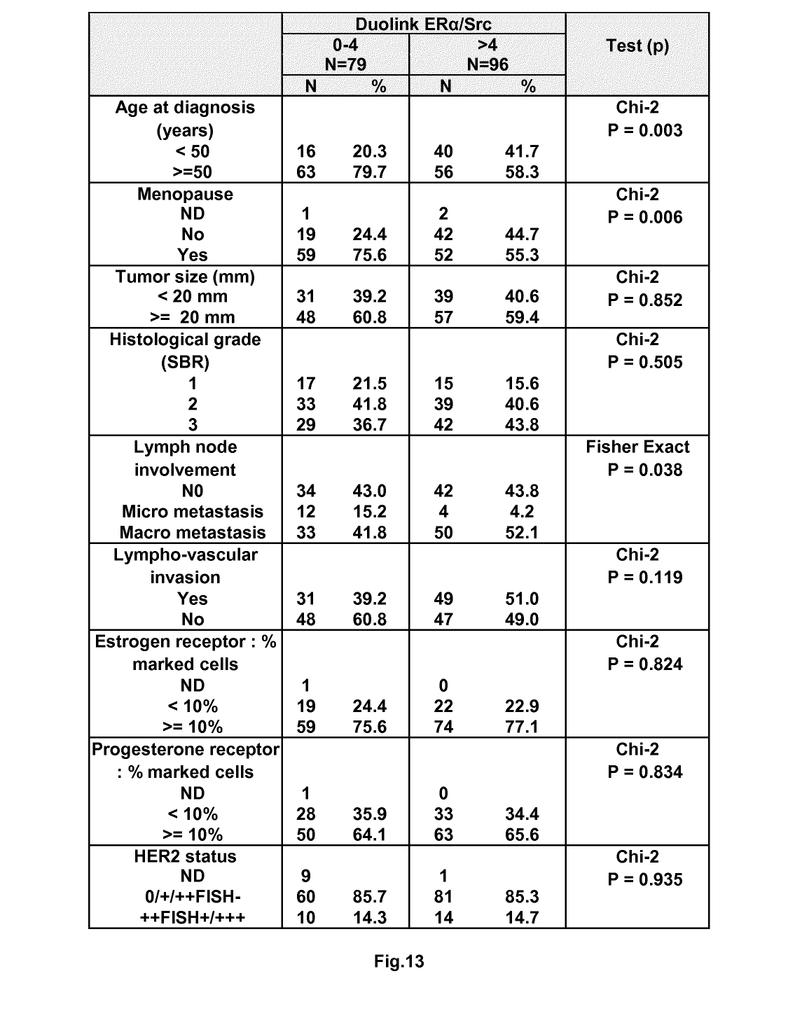

For the expression of ERα/Src, we did not find any association with the status of ERα, PR or HER2. However, age >50 years and menopausal status were significantly associated with a low expression of ERα/Src (respectively 80% and 76% vs 58% and 55% of patients with a high expression of ERα/Src, p=0.003 and p=0.006). ERα/Src expression was also associated with lymph node involvement (42% of patients with a low expression of ERα/Src had lymph node involvement vs. 52% of patients with a high expression of ERα/Src, p=0.038) (FIG. 13). Thus, a high expression of ERα/Src was associated with less favorable prognostic factors.

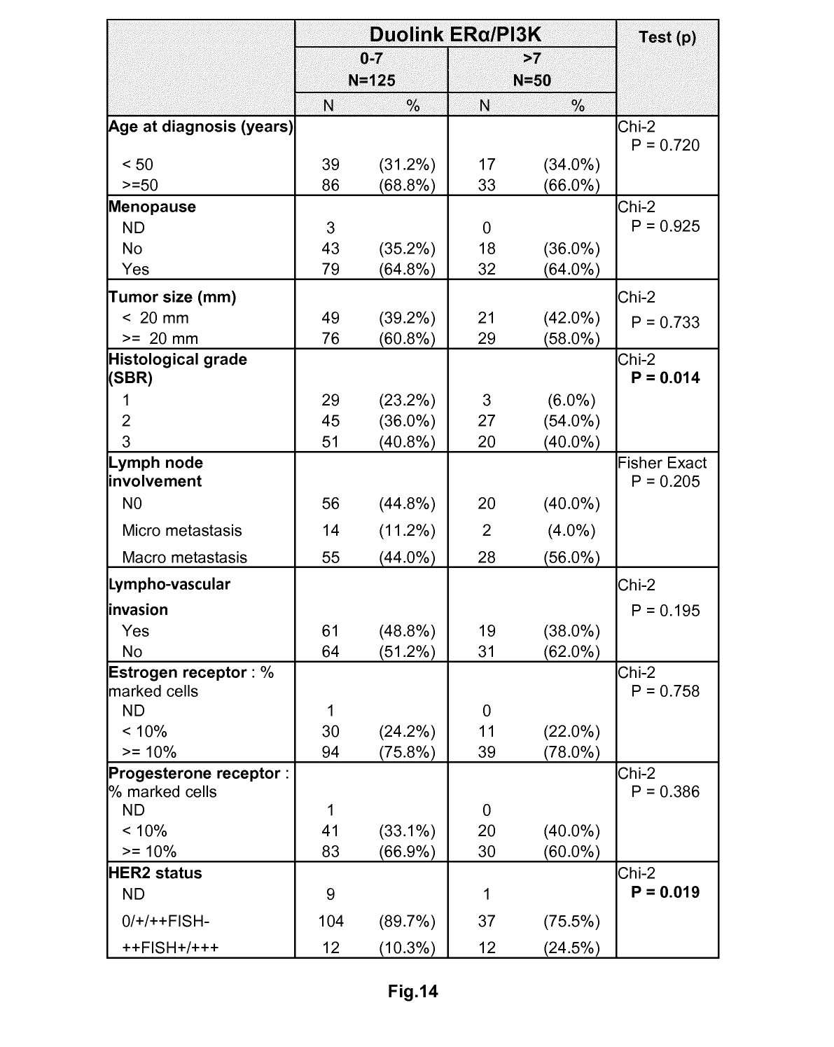

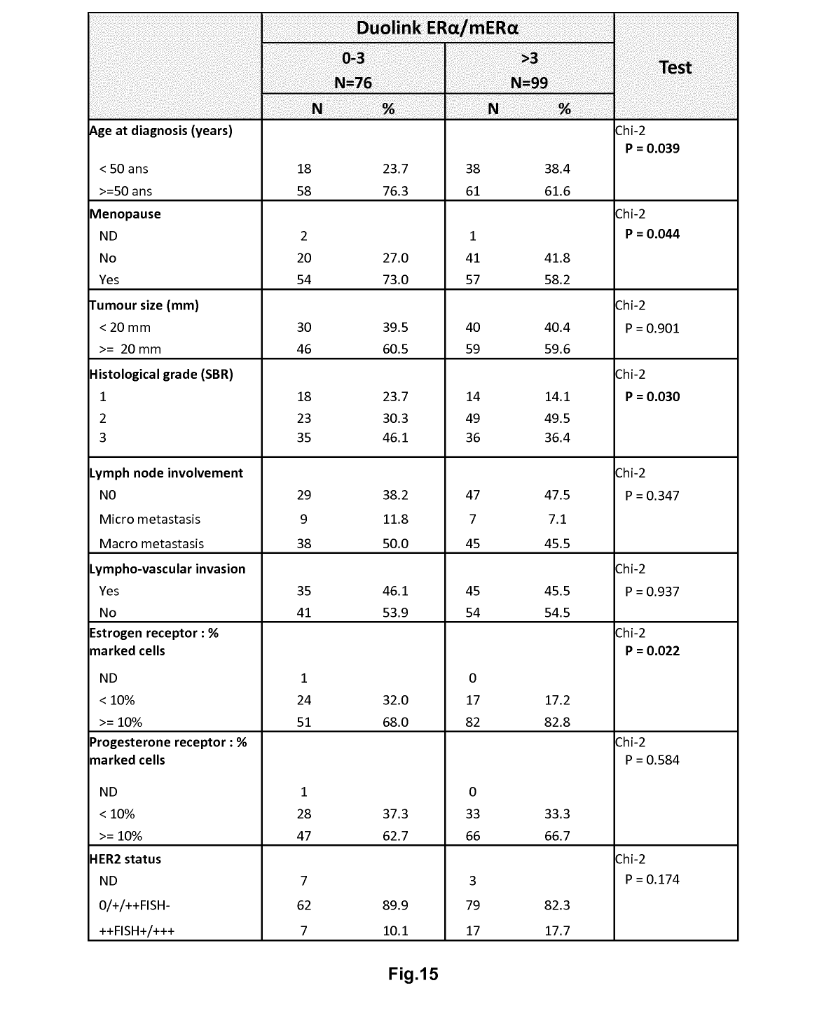

Regarding ERα/PI3K expression, we did not find any association with ERα or PR expression. However, a high expression of ERα/PI3K was associated with tumors overexpressing HER2 (25% of tumors with a high expression of ERα/PI3K overexpressed HER2 vs 10% of tumors with a low expression, p=0.019). Moreover, high expression of ERα/PI3K was associated with tumor grade, with more tumors presenting grade 2 or 3 when ERα/PI3K was highly expressed (p=0.014) (FIG. 14).
We found that high expression of mERα/ERα was significantly associated with youngest people (<50 years old), premenopausal status, higher grade SBR and ERα expression (FIG. 15).
Altogether, these data strongly suggest that estrogen nongenomic signaling is associated with common poor prognostic factors for breast cancer patients (Weigel and Dowsett, 2010).

### Survival Analysis and Predictive Value of ERα/Src and ERα/PI3K Interactions

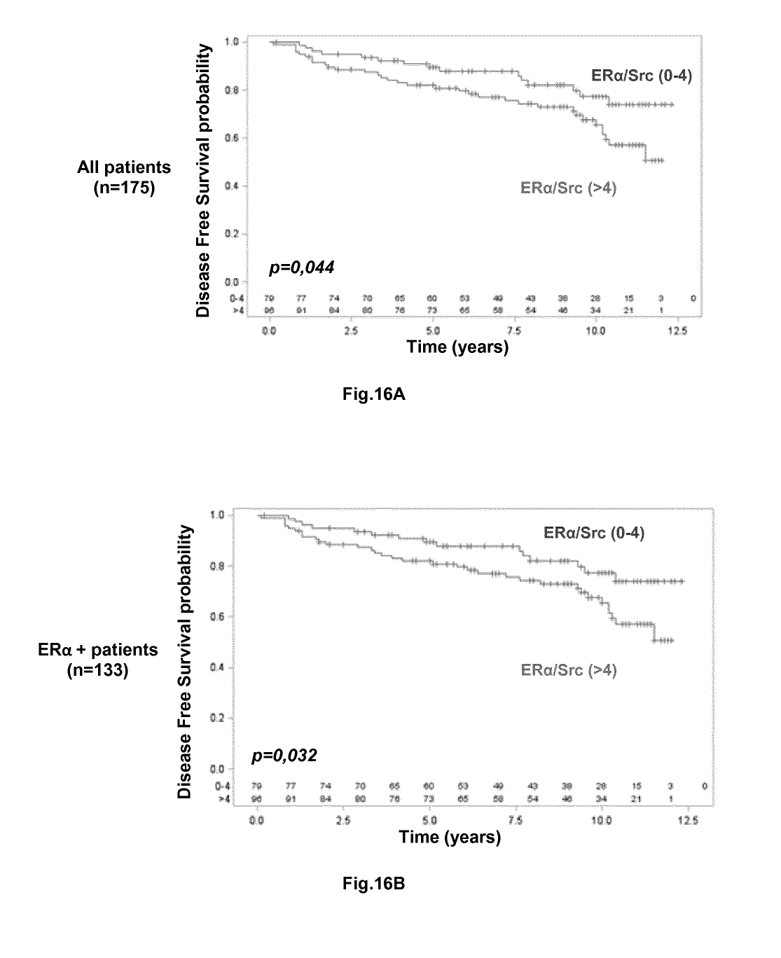

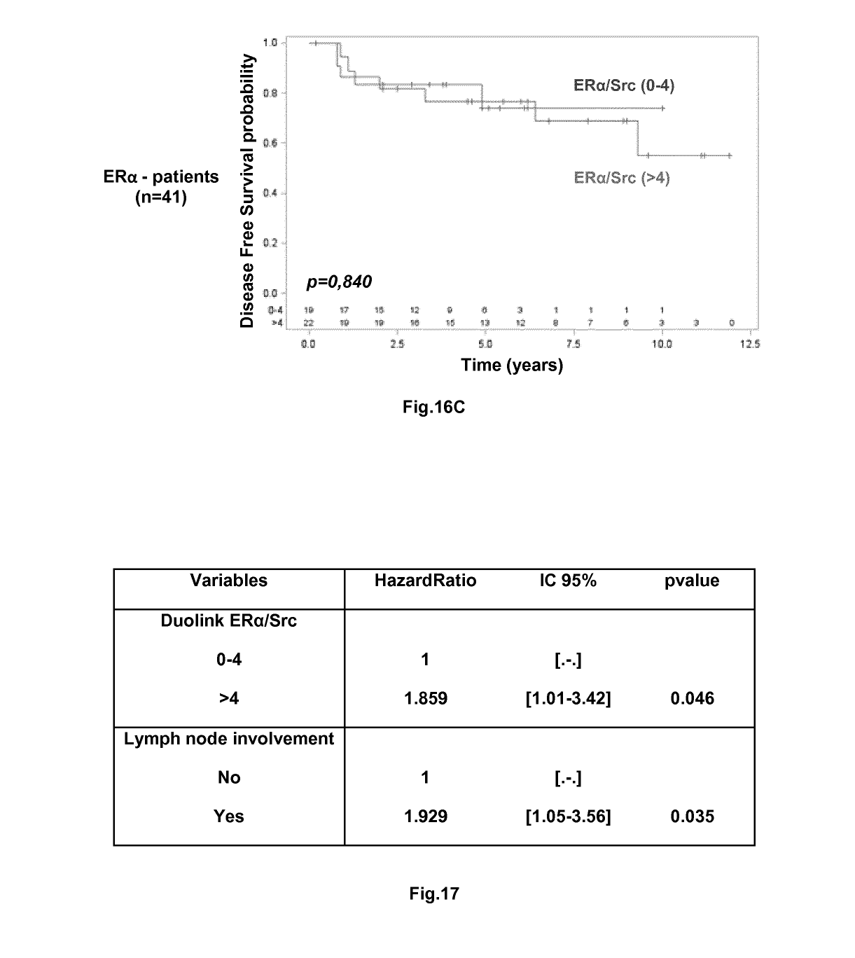

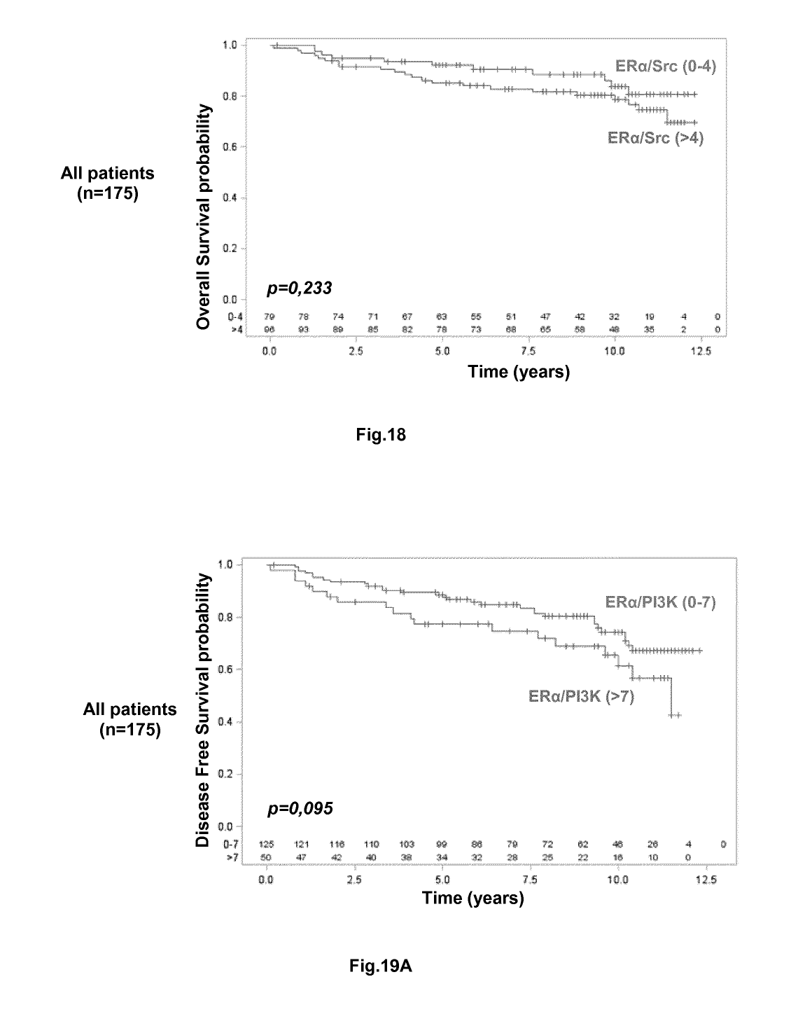

We next investigated how ERα/Src and/or ERα/PI3K expression were associated with patient outcomes. Regarding ERα/Src, high expression of this couple was associated with a decreased disease-free survival (DFS) (Log-Rank test, p=0.044) (FIG. 16A). Furthermore, within the subgroup of ERα-positive tumors, a high expression of ERα/Src was still associated with a reduced DFS (p=0.032) (FIG. 16B). For ERα negative tumors, the number of patients was probably not sufficient to conclude (FIG. 16C). In multivariate analysis, high expression of ERα/Src remained an independent prognostic factor (HR=1.86, 95% CI [1.01-3.42], p=0.047 adjusted on lymph node involvement (HR=1.93, 95% CI [1.05-3.56], p=0.035 (FIG. 17). Notice that parameters like SBR grade, ERα expression and lymph node involvement were not kept as independent prognostic factors in the final model. In terms of overall survival (OS), there was no statistical difference between tumors with high and low expression of ERα/Src (p=0.23) (FIG. 18).

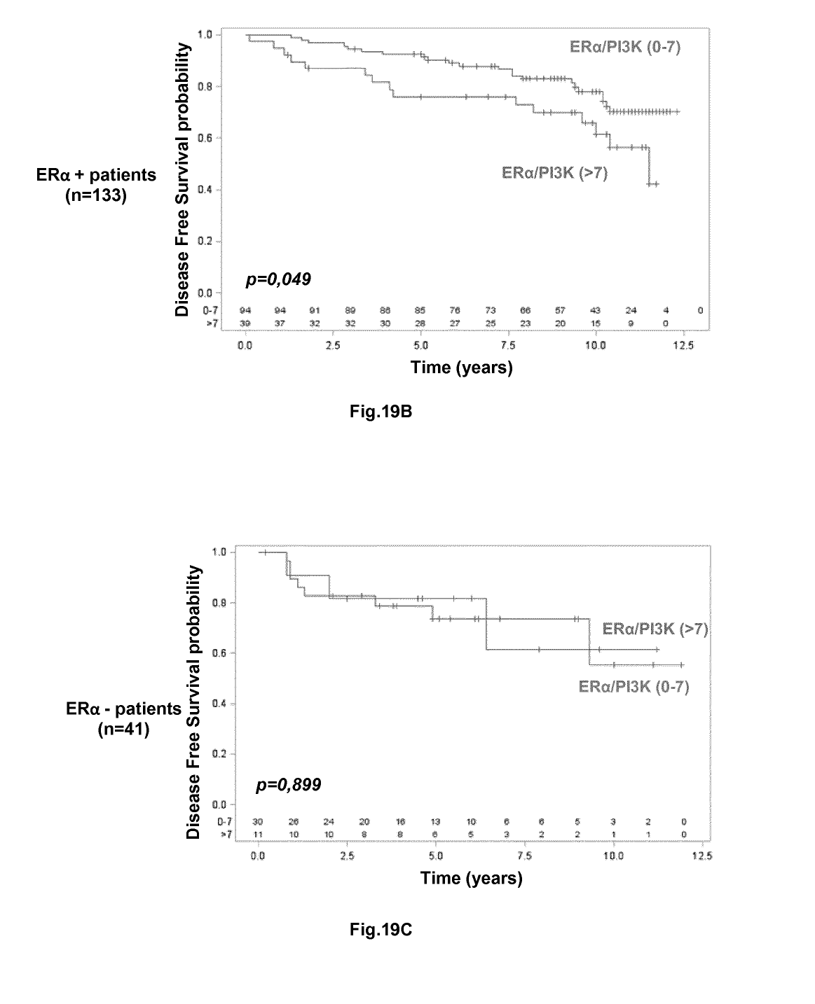

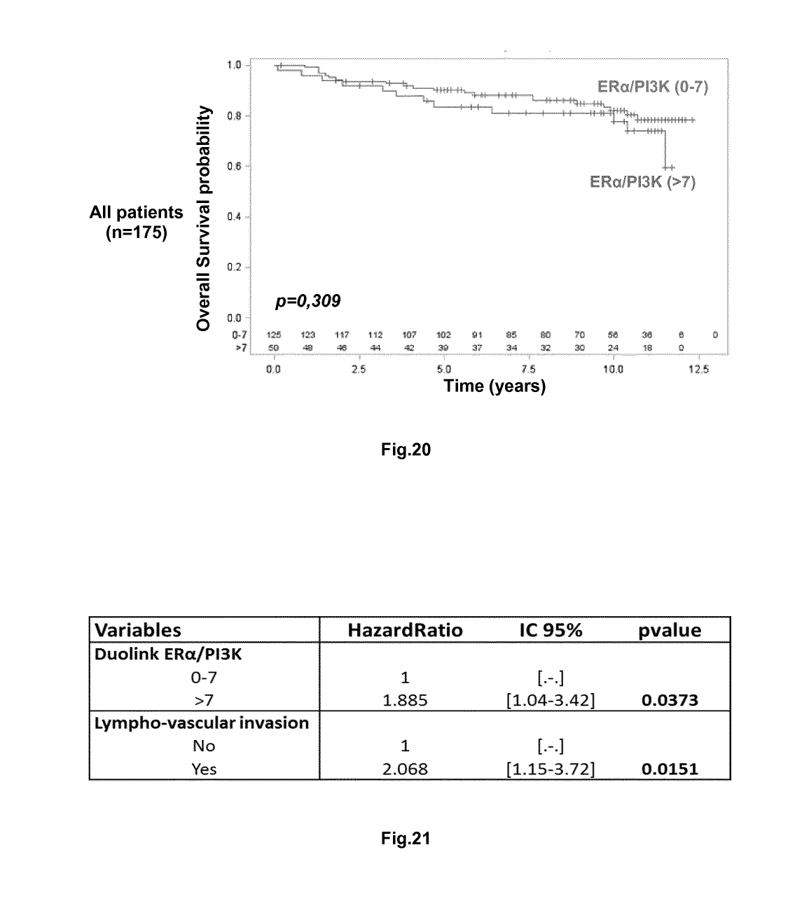

We made similar observations for the ERα/PI3K interaction. For all patients, we found no statistical association with either DFS or OS (p=0.096 and p=0.309 respectively), even though a tendency can be noticed on DFS's curves (FIGS. 19A and 21). However, for patients with ERα-positive tumors, expression of ERα/PI3K was a prognostic factor for DFS, with a worse prognosis for patients with tumors highly expressing ERα/PI3K, (Log-Rank test, p=0.049) (FIG. 19B). As for the ERα/Src interaction, the number of patients with ERα-negative tumors was too small to conclude (FIG. 19C). In multivariate analysis, high expression of ERα/PI3K was found to be linked with DFS (HR=1.89, 95% CI [1.04-3.42], p=0.0037) adjusted on lymph node involvement (HR=2.07, 95% CI [1.15-3.72], p=0.015) (FIG. 21).

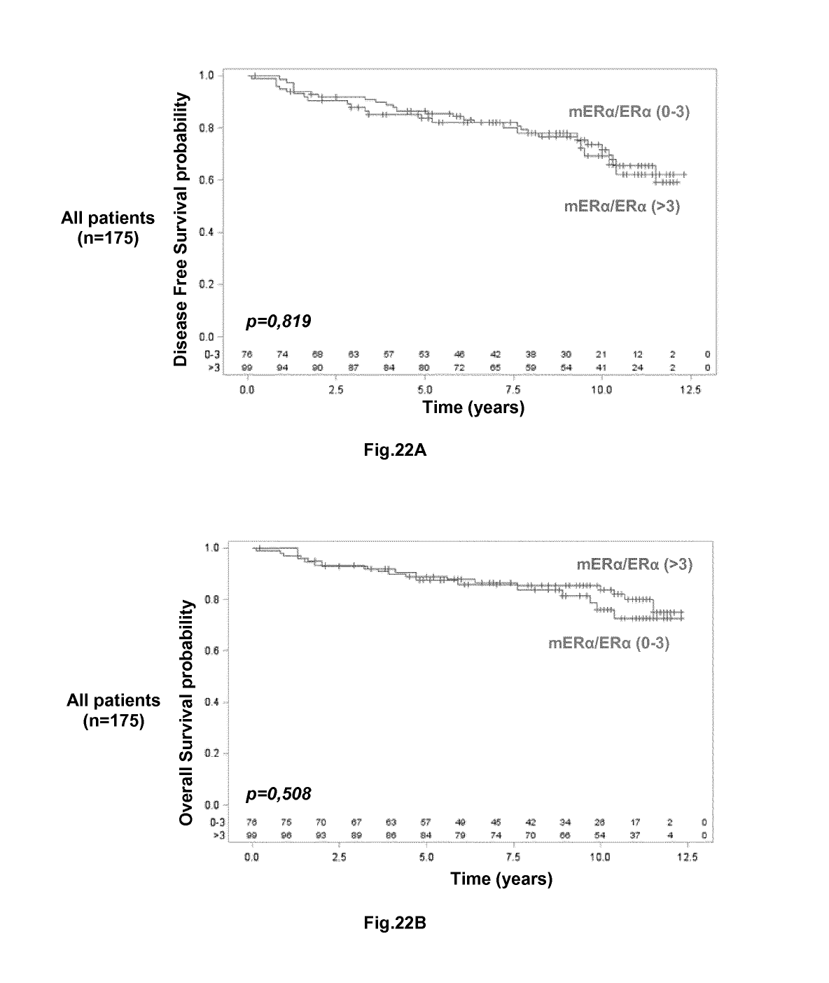

We did not find an association between ERα methylation and patient outcomes, as measured by the couple mERα/ERα. (FIGS. 22A, 22B).

### REFERENCES

Araujo J and Logothetis C (2010) Dasatinib: a potent SRC inhibitor in clinical development for the treatment of solid tumors. Cancer Treat Rev, 36, 492-500.
Le Romancer M., Poulard C, Cohen P, Sentis S, Renoir J M, and Corbo L (2011) Cracking the Estrogen Receptor's Posttranslational Code in Breast Tumors. Endocr Rev.
Le Romancer M., Treilleux I, Bouchekioua-Bouzaghou K, Sentis S, and Corbo L (2010) Methylation, a key step for nongenomic estrogen signaling in breast tumors. Steroids, 75, 560-564.
Le Romancer M., Treilleux I, Leconte N, Robin-Lespinasse Y, Sentis S, Bouchekioua-Bouzaghou K, Goddard S, Gobert-Gosse S, and Corbo L (2008) Regulation of estrogen rapid signaling through arginine methylation by PRMT1. Mol Cell, 31, 212-221.
Musgrove E A and Sutherland R L (2009) Biological determinants of endocrine resistance in breast cancer. Nat Rev Cancer, 9, 631-643.
Soderberg O, Gullberg M, Jarvius M, Ridderstrale K, Leuchowius K J, Jarvius J, Wester K, Hydbring P, Bahram F, Larsson L G, and Landegren U (2006) Direct observation of individual endogenous protein complexes in situ by proximity ligation. Nat Methods, 3, 995-1000.
Varricchio L, Migliaccio A, Castoria G, Yamaguchi H, de F A, Di D M, Giovannelli P, Farrar W, Appella E, and Auricchio F (2007) Inhibition of estradiol receptor/Src association and cell growth by an estradiol receptor alpha tyrosine-phosphorylated peptide. Mol Cancer Res, 5, 1213-1221.

1. A method for identifying a breast cancer likely to respond to treatment with anti-estrogens and to treatment with Src inhibitors and/or PI3K inhibitors or for determining the prognosis of a breast cancer in a patient comprising the following steps:
a. Measuring the level of expression of ERα/Src/PI3K protein complexes in the cytoplasm of cancer cells from a breast tumour sample previously taken from said patient,
b. Classifying the breast cancer as likely to respond to treatment with anti-estrogens and to treatment with Src inhibitors and/or PI3K inhibitors or as having a poor prognosis if ERα/Src/PI3K protein complexes are overexpressed in the cytoplasm of said cancer cells.

2. The method of claim 1 wherein in step b) the level of expression of ERα/Src/PI3K protein complexes is compared to the median level of expression of ERα/Src/PI3K protein complexes in healthy breast tissue.

3. The method of claim 1 wherein in step b) the level of expression of ERα/Src/PI3K protein complexes is compared to the median level of expression of ERα/Src/PI3K protein complexes in breast tumour samples.

4. The method of claim 1, wherein the breast cancer has been classified as ER+.

5. The method of claim 1, wherein the breast cancer has been classified as ER- and is classified as likely to respond to treatment with anti-estrogens and to treatment with Src inhibitors and/or PI3K inhibitors.

6. The method of claim 1 wherein the level of expression of ERα/Src/PI3K protein complexes is measured by detecting ERα/Src protein complexes by a Proximity Ligation assay using anti-ERα and anti-Src antibodies and/or by detecting ERα/PI3K protein complexes by a Proximity Ligation assay using anti-ERα and anti-PI3K antibodies.

7. A composition comprising anti-ERα and anti-Src antibodies and/or anti-ERα and anti-Src antibodies for its use in a method for determining the prognosis of a breast cancer in a patient or for identifying a breast cancer likely to respond to treatment with anti-estrogens and to treatment with Src inhibitors and/or PI3K inhibitors, by measuring the level of expression of ERα/Src/PI3K protein complexes in the cytoplasm of cancer cells from a breast tumour sample previously taken from said patient.

8. The composition of claim 7, wherein the level of expression of ERα/Src/PI3K protein complexes is measured by detecting ERα/Src protein complexes by a Proximity Ligation assay using anti-ERα and anti-Src antibodies and/or by detecting ERα/PI3K protein complexes by a Proximity Ligation assay using anti-ERα and anti-PI3K antibodies.

9. A composition comprising a Src inhibitor and/or a PI3K inhibitor for use in methods of treatment of a breast cancer patient, wherein said use comprises the following steps:
a. Measuring the level of expression of ERα/Src/PI3K protein complexes in the cytoplasm of cancer cells from a breast tumour sample previously taken from said patient,
b. Selecting a breast cancer patient having a breast cancer tumour in which the ERα/Src/PI3K protein complex is overexpressed in the cytoplasm of said cancer cells,
c. Administering to said patient a therapeutically effective amount of a Src inhibitor and/or a PI3K inhibitor.

10. The composition for use in methods of treatment of a breast cancer patient according to claim 9 wherein in step c) the Src inhibitor and/or the PI3K inhibitor are administered in combination with an anti-estrogen.

11. The composition for use in methods of treatment of a breast cancer patient according to claim 9, wherein in step a) the level of expression of ERα/Src/PI3K protein complexes is measured by detecting ERα/Src protein complexes by a Proximity Ligation assay using anti-ERα and anti-Src antibodies and/or the level of expression of ERα/Src/PI3K protein complexes is measured by detecting ERα/PI3K protein complexes by a Proximity Ligation assay using anti-ERα and anti-PI3K antibodies.

12. The composition for use in methods of treatment of a breast cancer patient according to claim 9, wherein the breast cancer has been classified as ER+.

13. The composition for use in methods of treatment of a breast cancer patient according to claim 9, wherein the breast cancer has been classified as ER−.

14. A method of treatment of a breast cancer patient in need thereof wherein said method comprises the following steps:
a. Selecting a breast cancer patient having a breast cancer tumour in which the ERα/Src/PI3K protein complex is overexpressed in the cytoplasm of said cancer cells,
b. Administering to said patient a therapeutically effective amount of Src inhibitor and/or PI3K inhibitor.

15. The method of claim 15 wherein in step c) the Src inhibitor and/or the PI3K inhibitor are administered in combination with an anti-estrogen.

16. The method of claim 14, wherein the breast cancer has been classified as ER+.

17. The method of claim 14, wherein the breast cancer has been classified as ER−.

18. The method of claim 14, wherein the patient is selected by measuring the level of expression of ERα/Src/PI3K protein complexes in the cytoplasm of cancer cells from a breast tumour sample previously taken from said patient by using a method according to claim 1.
